(19) United States
(12) Patent Application Publication
(10) Pub. No.: US 2015/0118282 A1
(43) Pub. Date: Apr. 30, 2015

(54) TRANSDERMALLY ABSORBABLE PREPARATION CONTAINING ROTIGOTINE

(71) Applicant: SK CHEMICALS CO., LTD., Seongnam-si (KR)

(72) Inventors: Hye-Min KIM, Anyang-si (KR); Yong-Youn Hwang, Suwon-si (KR); Won-No Youn, Seoul (KR); Yeo-Jin Park, Seoul (KR); Joon-Gyo Oh, Suwon-si (KR); Jong-Seob Im, Suwon-si (KR); Hun-Taek Kim, Seoul (KR)

(21) Appl. No.: 14/589,851

(22) Filed: Jan. 5, 2015

Related U.S. Application Data
(63) Continuation of application No. PCT/KR2013/006001, filed on Jul. 5, 2013.

(30) Foreign Application Priority Data
Jul. 5, 2012 (KR) 10-2012-0073295

(51) Int. Cl. A61K 47/12; A61K 47/32; A61K 9/00; A61K 31/381

(52) U.S. Cl. CPC A61K 47/12; A61K 31/381; A61K 47/32; A61K 9/0014
USPC 424/443; 514/438

(57) ABSTRACT

A method for preventing the precipitation of rotigotine crystals, including a step of mixing rotigotine and one or more crystallization prevention agents selected from the group consisting of fatty alcohols, fatty acids, fatty acid esters, fatty acid amides, and the derivatives thereof, and to a transdermally absorbable preparation that includes rotigotine and crystallization prevention agents.

### CROSS-REFERENCE TO RELATED APPLICATIONS

This application is a continuation of International Application No. PCT/KR2013/006001, filed on Jul. 5, 2013, and claims priority from and the benefit of Korean Patent Application No. 10-2012-0073295, filed on Jul. 5, 2012, each of which is hereby incorporated by reference for all purposes as if fully set forth herein.

### BACKGROUND

1. Field

Exemplary embodiments of the present invention relate to a method for preventing the crystallization of rotigotine, and more particularly, to a method for preventing the precipitation of rotigotine crystals, the method including a step of mixing rotigotine with at least one anti-crystallizer selected from the group that includes fatty alcohols, fatty acids, fatty acid esters, fatty acid amides, and derivatives thereof, and to a transdermally absorbable preparation containing rotigotine and anti-crystallizer.

2. Discussion of the Background

Rotigotine, (−)-5,6,7,8-tetrahydro-6-[propyl-[2-(2-thienyl)ethyl]amino]-1-naphtahlenol, is used for the treatment of Parkinson's disease (PD) and restless legs syndrome (RLS), as a non-ergoline dopamine agonist.

This rotigotine has been commercially available in a patch dosage form. A patch-type transdermal composition is disclosed in U.S. Pat. No. 7,413,747 B. Also, one of the commercially available patches employs a silicone adhesive, which is disclosed in EP 1033978B. However, the commercially available patches cause the precipitation of rotigotine crystals in room-temperature storage conditions, and thus it is difficult to secure the storage period for commercial distribution of the commercially available patches. In order to solve the problem, patent WO 2011/076879 discloses a rotigotine transdermal composition with an added polymer, such as polyvinylpyrrolidone.

Rotigotine transdermal composition products on the market contain polyvinylpyrrolidone, but polyvinylpyrrolidone serves to increase the viscosity of a drug storage layer. As a result, the coating may not be uniform, and the uniform mixing of the drug storage layer is difficult. Thus, measures capable of preventing crystallization to solve the problems are being constantly researched.

Rotigotine crystals may not be formed when a patch is prepared by mixing rotigotine with fatty alcohols, fatty acids, or the like.

The above information disclosed in this Background section is only for enhancement of understanding of the background of the invention and therefore it may contain information that does not form any part of the prior art nor what the prior art may suggest to a person of ordinary skill in the art.

### SUMMARY

Exemplary embodiments of the present invention provide a method for preventing the crystallization of rotigotine, the method including a step of mixing rotigotine with at least one anti-crystallizer selected from the group that includes fatty alcohols, fatty acids, fatty acid esters, fatty acid amides, and derivatives thereof.

Exemplary embodiments of the present invention provide a transdermally absorbable preparation containing, an active ingredient, at least one anti-crystallizer selected from the group that includes fatty alcohols, fatty acids, fatty acid esters, fatty acid amides, and derivatives thereof.

Exemplary embodiments of the present invention provide a method for preparing a transdermally absorbable preparation, the method including a step of mixing rotigotine with at least one material selected from the group that includes fatty alcohols, fatty acids, fatty acid esters, fatty acid amides, and derivatives thereof.

Exemplary embodiments of the present invention provide a transdermal therapeutic system, including: a drug-containing adhesive layer including rotigotine, an adhesive, and at least one anti-crystallizer selected from the group that includes fatty alcohols, fatty acids, fatty acid esters, fatty acid amides, and derivatives thereof; and a substrate supporting the drug-containing adhesive layer.

Additional features of the present invention will be set forth in the description which follows, and in part will be apparent from the description, or may be learned by practice of the present invention.

In accordance with exemplary embodiments of the present invention, there is provided a method for preventing the crystallization of rotigotine, the method including a step of mixing rotigotine with at least one anti-crystallizer selected from the group that includes fatty alcohols, fatty acids, fatty acid esters, fatty acid amides, and derivatives thereof.

In accordance with exemplary embodiments of the present invention, there is provided a transdermally absorbable preparation including, as active ingredients, rotigotine and at least one anti-crystallizer selected from the group that includes fatty alcohols, fatty acids, fatty acid esters, fatty acid amides, and derivatives thereof.

In accordance with exemplary embodiments of the present invention, there is provided a method for preparing a transdermally absorbable preparation, the method including a step of mixing rotigotine with at least one anti-crystallizer selected from the group that includes fatty alcohols, fatty acids, fatty acid esters, fatty acid amides, and derivatives thereof.

In accordance with exemplary embodiments of the present invention, there is provided a transdermal therapeutic system including: a drug-containing adhesive layer including rotigotine, an adhesive, and at least one anti-crystallizer selected from the group that includes fatty alcohols, fatty acids, fatty acid esters, fatty acid amides, and derivatives thereof and a substrate for supporting the drug-containing adhesive layer.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 illustrates images showing test results of an anti-crystallization effect according to the kind of anti-crystallizer of exemplary embodiments of the present invention (Comparative example 1: anti-crystallizer non-addition group, Example 1-1: oleic acid addition group, Example 1-2: caprylic acid addition group, and Example 1-3: hexanoic acid addition group).

FIG. 2 illustrates images showing test results of an anti-crystallization effect according to the proportion of anti-crystallizer of exemplary embodiments of the present invention (Comparative example 1: Anti-crystallizer non-addition group, Comparative example 2: 0.1% anti-crystallizer addition group, Example 2-1: 0.6% oleic acid addition group, Example 2-2: 5.9% oleic acid addition group, Example 2-3: 8.6% oleic acid addition group, and Example 2-4: 15.9% oleic acid addition group).

FIG. 3 illustrates images showing test results of the permeation into the human skin according to the proportion of anti-crystallizer (Comparative example 3: 37.0% anti-crystallizer addition group, Example 2-1: 0.6% oleic acid addition group, Example 2-2: 5.9% oleic acid addition group, Example 2-3: 8.6% oleic acid addition group, and Example 2-4: 15.9% oleic acid addition group).

### DETAILED DESCRIPTION OF THE ILLUSTRATED EMBODIMENTS

The present invention is described more fully hereinafter with reference to the accompanying drawings, in which exemplary embodiments of the present invention are shown. The present invention may, however, be embodied in many different forms and should not be construed as limited to the exemplary embodiments set forth herein. Rather, these exemplary embodiments are provided so that this disclosure is thorough, and will fully convey the scope of the present invention to those skilled in the art. In the drawings, the size and relative sizes of layers and regions may be exaggerated for clarity. Like reference numerals in the drawings denote like elements.

It will be understood that when an element or layer is referred to as being “on” or “connected to” another element or layer, it can be directly on or directly connected to the other element or layer, or intervening elements or layers may be present. In contrast, when an element is referred to as being “directly on” or “directly connected to” another element or layer, there are no intervening elements or layers present. It will be understood that for the purposes of this disclosure, “at least one of X, Y, and Z” can be construed as X only, Y only, Z only, or any combination of two or more items X, Y, and Z (e.g., XYZ, XYY, YZ, ZZ).

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Hereinafter, exemplary embodiments of the present invention will be described in detail.

Exemplary embodiments of the present invention provide a method for preventing the crystallization of rotigotine, the method including a step of mixing rotigotine with at least one anti-crystallizer selected from the group that includes fatty alcohols, fatty acids, fatty acid esters, fatty acid amides, and derivatives thereof.

The method for preventing the crystallization of rotigotine in exemplary embodiments of the present invention is characterized by including a step of mixing rotigotine with at least one anti-crystallizer selected from the group that includes fatty alcohols, fatty acids, fatty acid esters, fatty acid amides, and derivatives thereof.

Rotigotine ((−)-5,6,7,8-tetrahydro-6-[propyl-[2-(2-thienyl)ethyl]amino]-1-naphtahlenol) has a structure of chemical formula 1 below, and is used for the treatment of Parkinson's disease (PD) and restless legs syndrome (RLS), as a non-ergoline dopamine agonist.

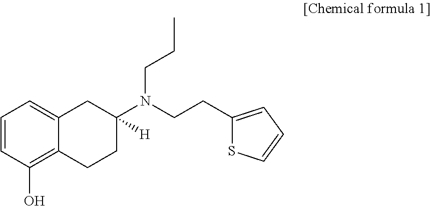 [SMILES: CCCN(CCc1cccs1)[C@H]1CCc2c(O)cccc2C1]

The anti-crystallizer of exemplary embodiments of the present invention is a material for suppressing the crystallization of rotigotine, and may be selected from the group that includes fatty alcohols, fatty acids, fatty acid esters, fatty acid amides, and derivatives thereof. The fatty alcohols, fatty acids, fatty acid esters, and fatty acid amides of the anti-crystallizer of exemplary embodiments of the present invention may include at least one carbon atom, and may include no more than 40 carbon atoms. The materials of this group may not include a carbon-carbon double bond, or may include at least one carbon-carbon double bond. The anti-crystallizer of exemplary embodiments of the present invention may be selected from the group that includes oleic acid, oleyl alcohol, caprylic acid (octanoic acid), and hexanoic acid.

The term “fatty alcohol” refers to any alcohol in which 8 to 22 carbon atoms are linked to each other in a chain shape. The fatty acids in exemplary embodiments of the present invention may be, for example, capryl alcohol, 2-ethyl hexanol, lauryl alcohol, tridecyl alcohol, myristyl alcohol, oleyl alcohol, and/or linoleyl alcohol.

The term “fatty acid” refers to a long, aliphatic chain with a carboxylic group, and includes both saturated fatty acids and unsaturated fatty acids. The fatty acids of the present invention may be, for example, myristoleic acid, palmitoleic acid, sapienic acid, oleic acid, and/or elaidic acid.

The term “fatty acid ester” refers to any ester resulting from the replacement of a hydrogen atom of fatty acid with an alkyl group, and the fatty acid esters of exemplary embodiments of the present invention may be, for example, isopropyl palmitate, isopropyl laurate, isooctylpalmitate, isooctyl stearate, lauryl stearate, butyl stearate, methyl n-caprylate, methyl n-caprate, methyl laurate, methyl stearate, and/or methyl oleate.

The term “fatty acid amide” refers to any amide resulting from the replacement of a hydrogen atom of fatty acid with an amide group, and the fatty acid amides of exemplary embodiments of the present invention may be, for example, oleamide, stearamide, erucamide, behenamide, N-oleylpalmitamide, and/or N-stearylerucamide.

The anti-crystallizer effectively blocks the crystallization of rotigotine, and enables the preparation of rotigotine-containing patches having high long-term storage stability. The fact that the crystallization of rotigotine is blocked when rotigotine is mixed with fatty alcohols, fatty acids, fatty acid esters, fatty acid amides, or the like is first presented in exemplary embodiments the present invention.

The amount of anti-crystallizer mixed with rotigotine is not particularly limited, but the weight ratio of rotigotine and the anti-crystallizer may be 10:0.1 to 40, 10:0.1 to 30, or 10:0.3 to 10:20. Less than 0.1 parts by weight of anti-crystallizer added may cause the crystallization of rotigotine, and more than 40 parts by weight of anti-crystallizer added may lower the permeability of rotigotine into the skin, resulting in a smaller therapeutic effect.

Exemplary embodiments of the present invention may be applied to various dosage forms including rotigotine as an active ingredient, and may be applied to a patch, a liquid, an ointment, and the like.

Exemplary embodiments of the present invention provide a transdermally absorbable preparation including rotigotine and an anti-crystallizer as active ingredients.

The transdermally absorbable preparation of exemplary embodiments of the present invention includes rotigotine as an active drug ingredient, can conveniently administer the drug into the human body in a skin patch manner, and can constantly maintain a drug effect for a long time. The transdermally absorbable preparation of exemplary embodiments of the present invention is not crystallized since it includes an anti-crystallizer as an active ingredient, and thus can be stably stored for a long time.

The rotigotine and anti-crystallizer, which are the active ingredients of the transdermally absorbable preparation of exemplary embodiments of the present invention, are as described as above.

The transdermally absorbable preparation of exemplary embodiments of the present invention may further include an adhesive in addition to the rotigotine and anti-crystallizer.

The adhesive allows the transdermally absorbable preparation of exemplary embodiments of the present invention to be in continuous contact with the skin and thus facilitates the absorption of the active ingredients, and may include an acryl based adhesive, a rubber based adhesive, an ethylene-vinyl acetate based adhesive, a styrene based adhesive, and a silicone based adhesive. The term “styrene based adhesive” refers to an adhesive including a styrene based bloc copolymer as a base material. The styrene based bloc copolymer may be at least one bloc copolymer selected from the group styrene-isoprene, styrene-butadiene, styrene-isoprene-styrene, styrene-butadiene-styrene, styrene-isoprene-styrene-styrene, and/or styrene-butadiene-styrene-butadiene, but is not limited thereto.

The adhesive of exemplary embodiments of the present invention is contained at a ratio of 40 to 500 parts by weight per 10 parts by weight of rotigotine.

The transdermally absorbable preparation of exemplary embodiments of the present invention may further include a skin permeation promoter, a vehicle, an abirritant, or a carrier.

The skin permeation promoter is for promoting the permeation of a drug into the skin, and may include, for example, hydrophilic organic solvent, aprotic solvent, fatty acid, fatty alcohol, fatty acid ester, pyrrolidone, essential oil, surfactant, phospholipids, and/or the like.

The carrier contained in the transdermally absorbable preparation of exemplary embodiments of the present invention is a biocompatible carrier, and may be, but is not limited to, a carrier for parenteral administration. The carrier for parenteral administration may include water, appropriate oil, a saline solution, aqueous glucose, glycol, and the like. For other pharmaceutically acceptable carriers, one disclosed in the following document may be referenced (Remington's Pharmaceutical Sciences, 19th ed., Mack Publishing Company, Easton, Pa., 1995). The concentration of the biocompatible carrier may be, but is not limited to, about 2 to 70 wt %.

The transdermally absorbable preparation according to exemplary embodiments of the present invention may be formulated into a patch, a liquid, an ointment, or the like.

Further, exemplary embodiments of the present invention provide a method for preparing a transdermally absorbable preparation, the method including a step of mixing rotigotine with at least one anti-crystallizer selected from the group that includes fatty alcohols, fatty acids, fatty acid esters, fatty acid amides, and derivatives thereof.

The method of exemplary embodiments of the present invention includes a step of mixing rotigotine with at least one anti-crystallizer selected from the group that includes fatty alcohols, fatty acids, fatty acid esters, fatty acid amides, and derivatives thereof. The rotigotine and anti-crystallizer are as described above.

Further, the preparing method of exemplary embodiments of the present invention may include a step of mixing an adhesive with a mixture of rotigotine and the anti-crystallizer.

According to the preparing method of exemplary embodiments of the present invention, the rotigotine crystals are not precipitated, and thus a transdermally absorbable preparation having significantly improved long-term stability can be prepared.

Exemplary embodiments of the present invention provides a transdermal therapeutic system including: a drug-including adhesive layer including rotigotine, an adhesive, and at least one anti-crystallizer selected from the group that includes fatty alcohols, fatty acids, fatty acid esters, fatty acid amides, and/or derivatives thereof; and a substrate supporting the drug-containing adhesive layer.

The transdermal therapeutic system of exemplary embodiments of the present invention is characterized by including a drug-containing adhesive layer including rotigotine, an adhesive, and at least one anti-crystallizer selected from the group that includes fatty alcohols, fatty acids, fatty acid esters, fatty acid amides, and derivatives thereof; and a substrate supporting the drug-containing adhesive layer, and has a therapeutic effect against Parkinson's disease and restless legs syndrome through the permeation of rotigotine into the skin.

The drug-containing adhesive layer of exemplary embodiments of the present invention is characterized by including rotigotine as an active drug, an anti-crystallizer for preventing the precipitation of rotigotine crystals, and an adhesive. The rotigotine, anti-crystallizer, and adhesive of exemplary embodiments of the present invention are as described as above.

In the drug therapeutic system of exemplary embodiments of the present invention, the content of anti-crystallizer may be, but is particularly not limited to, more than 0.1 wt % to no more than 37 wt % based on the total weight of the drug-containing adhesive layer. If no more than 0.1 wt % of anti-crystallizer is added, it may be highly likely that rotigotine will be crystallized within four weeks, and if more than 37 wt % of anti-crystallizer is added, it may lower the skin permeability of the rotigotine.

In the drug-containing adhesive layer of exemplary embodiments of the present invention, the ratio of rotigotine, the anti-crystallizer, and the adhesive may not be particularly limited, and the weight ratio of the rotigotine, the anti-crystallizer, and the adhesive may be 10:0.1 to 40:40 to 500. The proportion of the anti-crystallizer may be 0.3 to 20.

In cases where the proportion of the adhesive is lower than the above numerical range, the adhesive strength deteriorates and thus the drug-containing adhesive layer does not adhere to the skin. In cases where the proportion of the adhesive is higher than the above numerical range, the drug-containing adhesive layer adheres to the skin too firmly, and thus when the drug delivery system is attached to and detached from the skin, the skin may be irritated and the drug release may be delayed.

The transdermal therapeutic system of exemplary embodiments of the present invention is characterized by including a substrate for supporting the drug-containing adhesive layer. For the substrate, any substrate that can be used for the patches or the like may be used, and any substrate which is thin and flexible, has no reactivity with the drug-containing adhesive layer, and has no reactivity with the skin, causing no allergic reaction, is used as the substrate of exemplary embodiments of the present invention. For example, a non-woven fabric, polyethylene terephthalate, soft polyvinylchloride, polyurethane, polyester, and/or a silicone coating film may be used as the substrate in exemplary embodiments of the present invention. A silicone coating film or polyethylene terephthalate (PET) may be used.

In the transdermal therapeutic system of exemplary embodiments of the present invention, the content of rotigotine may vary depending on the age, body weight, and gender of a patient, the manner of administration, the health condition, and the degree of severity of a disease, and may be applied once a day or divided into multiple doses such that 0.5 to 100 mg of an active material can be administered.

In an exemplary embodiment of exemplary embodiments of the present invention, the drug-containing adhesive layer was prepared by mixing rotigotine, ethanol, hexane, polyisobutylene, and an anti-crystallizer, coated on a silicone coating film, and then bonded to a PET film, thereby manufacturing a transdermal therapeutic system. The transdermal therapeutic system was tested with respect to stability thereof by being stored for two weeks or four weeks in accelerated test conditions. As a result, it was confirmed that rotigotine crystals were precipitated in a control group not containing anti-crystallizer, and the rotigotine crystals were not precipitated in the transdermal therapeutic system of exemplary embodiments of the present invention.

In an exemplary embodiment of the present invention, the stability of rotigotine according to the change in the content of the anti-crystallizer was measured by varying the content of the anti-crystallizer contained in the transdermal therapeutic system. As a result, it was confirmed that the rotigotine crystals were not precipitated within a range of more than 0.1 wt % but no more than 37 wt % of the anti-crystallizer based on the total weight of the drug-containing adhesive layer. It can be seen that the weight ratio of rotigotine and the anti-crystallizer is 10:0.1 to 10:40 in terms of the wt %. The weight ratio thereof may be 10:0.1 to 10:30.

As set forth above, exemplary embodiments of the present invention provide a method for preventing the crystallization of rotigotine, a transdermally absorbable preparation including rotigotine and an anti-crystallizer as active ingredients, a method for preparing the same, and a transdermal therapeutic system including a drug-containing adhesive layer including an anti-crystallizer, rotigotine, and an adhesive, and a substrate. The method and system of exemplary embodiments of the present invention prevent the precipitation of rotigotine, thereby increasing the long-term storage stability, and thus can be effectively applied to the manufacture of rotigotine-containing patches or the like.

Hereinafter, exemplary embodiments of the present invention will be described in detail with reference to the following examples. However, the following examples are merely for illustrating exemplary embodiments of the present invention, and are not intended to limit the scope of the present invention.

### Example 1

### Manufacture of Patches Containing Anti-Crystallizers and Evaluation on Crystallization Therein

Rotigotine transdermal preparations with three kinds of anti-crystallizers were prepared following table 1 below.

TABLE 1

| Classification | Kind of anti-crystallizer |
|---|---|
| Comparative example 1 | Not added |
| Example 1-1 | Oleic acid |
| Example 1-2 | Caprylic acid |
| Example 1-3 | Hexanoic acid |

For comparative example 1, 300 mg of rotigotine was mixed with and completely dissolved in 600 mg of ethanol, and then 1 g of hexane was added thereto, thereby preparing a homogenous solution. For examples 1-1 to 1-3, 300 mg of rotigotine was mixed with and completely dissolved in 300 mg of ethanol and 300 mg of anti-crystallizers, and then 1 g of hexane was added thereto, thereby preparing homogenous solutions. 4.2 g of a styrene based adhesive was put into these solutions, followed by mixing for 10 minutes at 600 rpm or higher, thereby preparing transdermal absorbable preparations. The prepared transdermal absorbable preparations were allowed to stand at room temperature, thereby allowing bubbles to burst. After confirming the removal of bubbles, the mixtures without bubbles (transdermal absorbable preparations) were coated on silicone coating films such that rotigotine can be contained at 0.4-0.5 mg per unit area (1 cm²), and then polyester (PET) films were bonded thereto, thereby manufacturing patches. Each of the manufactured transdermal administration systems (patches) was molded into a circular shape of 10 cm², which was then put in a Petri dish, and then stored in accelerated test conditions of a temperature of 40° C. and a relative humidity of 60% (for 2 weeks or 4 weeks). The precipitation of crystals was observed by the naked eye and photographed by a digital camera.

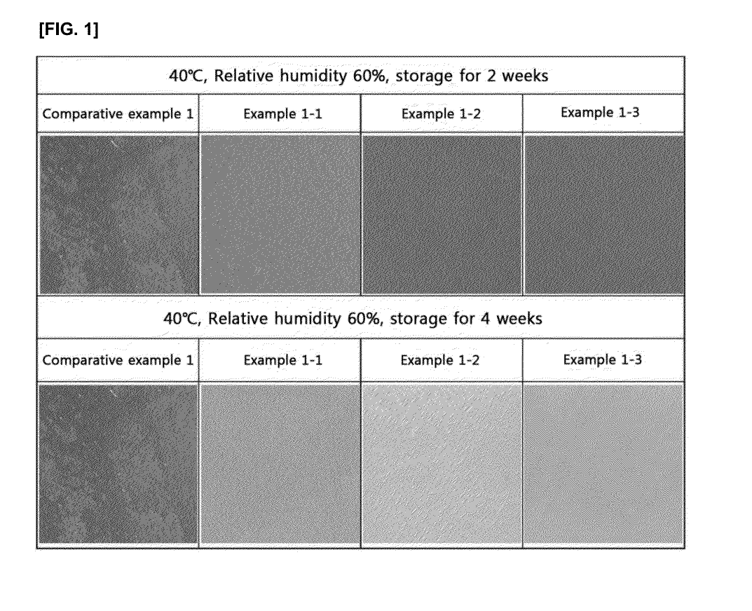

As a result of testing, as shown in [FIG. 1], under accelerated test conditions of a temperature of 40° C. and a relative humidity of 60%, the precipitation of crystals was observed in comparative example 1 with no added anti-crystallizer, but the precipitation of crystals was not observed in examples 1-1 to 1-3 with added fatty acids in spite of storage for 2 weeks or 4 weeks. Therefore, it can be confirmed that the storage ability of the rotigotine transdermal preparation can be improved by adding an anti-crystallizer.

### Example 2

### Manufacture of Patches for Different Proportions of Anti-Crystallizers and Evaluation on Crystallization Therein

The crystallizing degree of rotigotine according to the proportion of anti-crystallizer was measured.

Patches were manufactured following ingredient contents listed on table 2. Specifically, by the same method as in example 1, a drug-containing matrix layer (rotigotine content: 0.4-0.5 mg/cm²), in which rotigotine, oleic acid, and a styrene based adhesive (Durotak 87-6911 (Henkel)) were coated on a silicone coating film, was formed, and then bonded to a support film of a polyester material, thereby manufacturing a patch coated with a transdermally absorbable preparation. The manufactured patch was molded into a circular shape of 10 cm², which was then put in a Petri dish, and then stored in accelerated test conditions of a temperature of 40° C. and a relative humidity of 60% (for 2 weeks). The precipitation of crystals was observed by the naked eye and photographed by a digital camera.

TABLE 2

| Solid content | Comp. | Comp. | Comp. | Example | Example | Example | Example |
|---|---|---|---|---|---|---|---|
| (%) | example 1 | example 2 | example 3 | 2-1 | 2-2 | 2-3 | 2-4 |
| Rotigotine | 9.4 | 17.5 | 1.2 | 17.4 | 8.9 | 8.6 | 7.9 |
| Oleic acid | 0.0 | 0.1 | 37.0 | 0.6 | 5.9 | 8.6 | 15.9 |
| Adhesive | 90.6 | 82.4 | 61.7 | 82.0 | 85.2 | 82.8 | 76.2 |

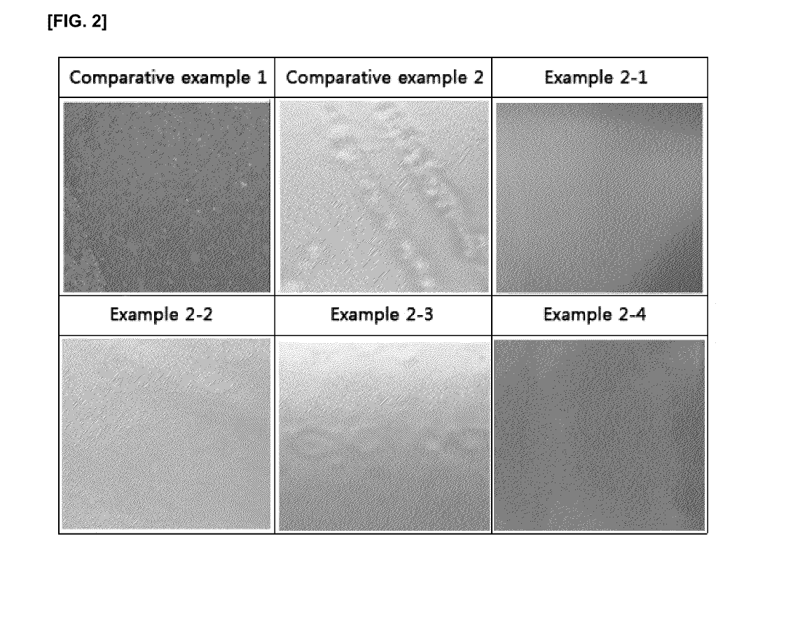

As can be seen from test results of FIG. 2, the crystals were precipitated while the transdermally absorbable preparation with no added anti-crystallizer (comparative example 1) was stored, and crystals of an active material were observed within 4 weeks after storage under conditions of a temperature of 40° C. and a relative humidity of 60% in comparative example 2 in which oleic acid is contained in 0.1% based on the total solid content of the patch. However, crystals of an active material were not observed during the storage for 4 weeks or longer in the transdermally absorbable preparations with 0.6% or more of oleic acid (examples 2-1 to 2-4 in table 2). Therefore, the transdermally absorbable preparation containing an anti-crystallizer of exemplary embodiments of the present invention does not cause crystallization even during the distribution at room temperature, thereby significantly improving the storage convenience.

### Example 3

### In Vitro Permeation Test on Patch According to Proportion of Anti-Crystallizer

In vitro skin permeation test was conducted on the transdermally absorbable preparations prepared in example 2 using Franz cells (vertical diffusion cells, Hanson research). Each of the prepared transdermally absorbable preparations were molded into a size of 1 cm², which was then fixed on human cadaver skin, and then the test liquid inside the Franz cells, which passed through the skin from the transdermally absorbable preparation and diffused in the skin, was automatically collected every hour. The collected test liquid was pre-treated through centrifugation, and then was analyzed through high-performance chromatography.

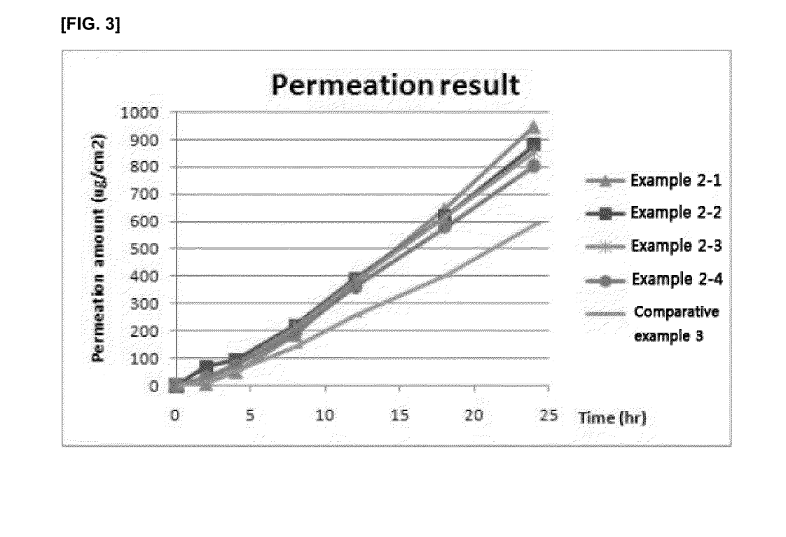

The results are shown in FIG. 3, and confirmed the skin permeation of rotigotine according to the proportion of anti-crystallizer. The transdermally absorbable preparation, in which the proportion of oleic acid is 37% in the entire preparation (comparative example 3), showed relatively low rotigotine skin permeation The lower proportion of anti-crystallizer tends to show a relatively rapid rotigotine release pattern.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A method for preventing the crystallization of rotigotine, the method comprising:
mixing rotigotine with at least one anti-crystallizer selected from the group consisting of fatty alcohols, fatty acids, fatty acid esters, fatty acid amides, and derivatives thereof.

2. The method of claim 1, wherein the rotigotine and the anti-crystallizer are mixed at a weight ratio of 10:(0.1 to 40).

3. The method of claim 2, wherein the rotigotine and the anti-crystallizer are mixed at a weight ratio of 10:(0.1 to 30).

4. A transdermally absorbable preparation, comprising:
active ingredients comprising rotigotine and at least one anti-crystallizer selected from the group consisting of fatty alcohols, fatty acids, fatty acid esters, fatty acid amides, and derivatives thereof.

5. A method for preparing a transdermally absorbable preparation, the method comprising:
mixing rotigotine with at least one anti-crystallizer selected from the group consisting of fatty alcohols, fatty acids, fatty acid esters, fatty acid amides, and derivatives thereof.

6. A transdermal therapeutic system, comprising:
a drug-containing adhesive layer comprising rotigotine, an adhesive, and at least one anti-crystallizer selected from the group consisting of fatty alcohols, fatty acids, fatty acid esters, fatty acid amides, and derivatives thereof; and
a substrate configured to support the drug-containing adhesive layer.

7. The system of claim 6, wherein the adhesive is selected from the group consisting of a silicone based adhesive, a rubber based adhesive, an acryl based adhesive, and an ethylene-vinyl acetate based adhesive.

8. The system of claim 7, wherein the rubber based adhesive is a styrene based adhesive.

9. The system of claim 6, wherein the amount the anti-crystallizer is in a range from 0.1 wt % to 37 wt % based on the total weight of the drug-containing adhesive layer.

10. The system of claim 6, wherein the drug-containing adhesive layer comprises the rotigotine, the anti-crystallizer, and the adhesive at a weight ratio of 10:(0.1 to 40):(40 to 500).
